(19) United States
(12) Patent Application Publication
(10) Pub. No.: US 2011/0280762 A1
(43) Pub. Date: Nov. 17, 2011

(54) METHOD IN THE TREATMENT OF ODOROUS GASES OF A CHEMICAL PULP MILL

(75) Inventors: Esko Mattelmäki, Porvoo (FI); Kari Saviharju, Espoo (FI)

(73) Assignee: ANDRITZ OY, Helsinki (FI)

(21) Appl. No.: 12/675,249

(22) PCT Filed: Aug. 20, 2008
(86) PCT No.: PCT/FI08/00094

§ 371 Date: Jan. 31, 2011

(30) Foreign Application Priority Data
Sep. 3, 2007 (FI) 20070671

(51) Int. Cl. A61L 9/16

(52) U.S. Cl. 422/5; 422/169

(57) ABSTRACT

A method for treating odorous gases of a chemical pulp mill, according to which method odorous gases are combusted in a separate combustion device and flue gas generated therein is scrubbed. The scrubbed flue gas is led into a recovery boiler. The flue gas is scrubbed in a series of scrubbers wherein scrubbing solution discharged from one scrubber may be fed to a preceding scrubber.

The present invention relates to a method for treating odorous gases of a chemical pulp mill and for improving the control of nitrogen oxide emissions.

In sulfate pulping, wood is treated in white liquor containing sodium hydroxide and sodium sulfide, whereby the lignin is hydrolyzed. Thereby several organic sulfur compounds are formed, such as methylmerkaptan, dimethylsulfide and dimethyldisulfide. These very compounds together with hydrogen sulfide cause the unpleasant smell of exhaust gases of chemical pulp mills. These gases are formed in several stages of a chemical pulping process, such as at the digester plant and the waste liquor evaporation. Malodorous sulfur compounds are removed most usually by collecting the malodorous gases from various sources and by combusting them either in a lime kiln, a chemical recovery boiler or a separate combustion apparatus. During combustion all sulfur-containing substances are oxidized to sulfur dioxide, sulfur trioxide, and, in the presence of alkali, also to sodium sulfate, and they are passed into flue gases.

In addition to sulfur compounds, digestion generates also methanol and ammonia. Vapors containing sulfur compounds, ammonia and methanol are released abundantly for instance in black liquor evaporation, where said compounds are distilled and condensed into condensates of a multistage evaporation plant. Foul condensates are usually purified in a steam stripper, where the condensate and steam are put into contact with each other and impurities are transferred from the condensate into the steam, while the condensate stream is obtained in purified form for further use. The exhaust vapor from the stripper is led via a post-condenser to combustion or directly to methanol liquefaction. Non-condensable gases (NCG) are combusted together with the flow of other odorous gases of the mill.

The odorous gases are typically divided into strong odor gases (LVHC Low Volume High Concentration) and dilute odorous gases (HVLC, High Volume Low Concentration). The strong odorous gases originate mainly from the digester plant, the evaporation plant or stripping. Dilute odorous gases are collected from containers and devices from the fiber line, evaporation plant, tall oil plant and causticizing plant. Dilute odorous gases contain the same components as the strong odorous gases, but they also contain so much air that the concentrations are remarkably lower.

The purpose of odorous gas combustion is to oxidize the reduced sulfur compounds contained in the gas, such as hydrogen sulfide, sulfur dioxide, and therefore the combustion is to take place in the presence of a remarkable volume of excess air (e.g. approximately 3-4%) and at a high temperature. Thereby the ammonia contained in the odorous gas is in its turn oxidized into nitrogen oxides. Especially the strong odorous gases contain nitrogen compounds, so that their combustion specifically has an influence on the nitrogen oxide emissions of the mill.

Finnish patent publication 105215 discloses a method, in which ammonia is removed from odorous gases prior to their combustion, whereby the nitrogen oxide content of the flue gas generated in the combustion can be significantly reduced. Preferably the ammonia is removed by scrubbing said gases in order to bind the ammonia off them. The scrubbing solution can preferably be a bisulfite solution originating from the scrubbing of flue gases formed in the combustion of the gases. Some other applicable solution originating from the chemical pulp mill and having a pH in the neutral or acid range, such as acid bleaching effluent or waste acid from the chlorine dioxide plant can also be used.

In view of the detrimental nitrogen compound emissions of the chemical pulp mill, a specific problem may be separate combustion of strong odorous gases.

An object of the present invention is to minimize the nitrogen oxide emissions of the flue gases of odorous gas combustion. A specific object of the invention is to provide a method for controlling the emissions of detrimental nitrogen compounds, especially nitrogen oxides, from a chemical pulp mill in a way that is more efficient than the prior methods when practicing separate combustion of odorous gases.

For achieving these goals the present invention relates to a method, in which odorous gases of a chemical pulp mill are combusted in a separate combustion device and flue gas generated therein is scrubbed. The method is characterized in that the scrubbed flue gas is led into a chemical recovery boiler.

An advantage of the invention in this regard is that the nitrogen oxides (NOx) in the flue gases of the separate combustion are not released into the atmosphere. The NOx-content of the recovery boiler flue gases does not increase substantially or at all, although the flue gas from the odorous gas combustion is fed into the boiler.

In the method according to the invention, especially strong odorous gases are treated, which are combusted in a way known per se in a separate combustion device, such as a fire tube boiler. In this boiler, the fuel and combustion air are typically fed in via one end of a typically horizontal tubular boiler space and the flue gases generated in the combustion are discharged via the opposite end of the boiler. Preferably this kind of a boiler is provided with a separate odorous gas burner, where the strong odorous gases are combusted.

The flue gas generated in the odorous gas combustion device is scrubbed for removing sulfur compounds. According to a preferred embodiment, the flue gas is scrubbed in at least two stages. In the first stage the flue gas is scrubbed with a sodium hydroxide-containing solution, whereby sodiumbisulfite (NaHSO₃) is generated. Bisulfite is required at a chemical pulp mill, e.g. in the pulp bleaching plant in destroying bleaching chemical residuals, such as chlorine dioxide residuals. In the first flue gas scrubbing stage, the amount of bisulfite needed at the mill can advantageously be produced for a specific purpose.

The next scrubbing stage comprises removing from the flue gas sulfur compounds, such as sulfur dioxide, formed in the combustion, whereby the scrubbing solution is preferably oxidized white liquor. The desulfuration stage is preferably carried out in two scrubbers. Fresh scrubbing solution is led in the flue gas flow direction into the latter scrubber, wherefrom the scrubbing solution is further led to a preceding scrubber. In the desulfuration, the sulfur oxides of the flue gas react into sulfites, and the scrubbing solution containing the sulfites is led into the chemical cycle of the mill, for instance via a white liquor tank.

The scrubbed cooled flue gas is led into a recovery boiler. According to an embodiment, the scrubbed flue gas is led into a burner mounted in the recovery boiler, which burner also receives air and preferably methanol and if required, other substance in addition to the flue gas. The burner can be a device similar to a typical odorous gas burner. It can be located at the secondary air level in the recovery boiler.

According to another embodiment, the scrubbed flue gas coming from the odorous gas combustion can be led directly into the recovery boiler, for instance via the air ports for combustion air, in a way similar to the leading of dilute odorous gases to the recovery boiler as combustion air. The flow rate of the scrubbed flue gas is so low compared to e.g. the combustion air amount of the recovery boiler that this kind of introduction thereof into the recovery boiler does not deteriorate the operation of the boiler.

The invention is described in more detail in the appended drawing, which illustrates schematically a preferred embodiment of the invention.

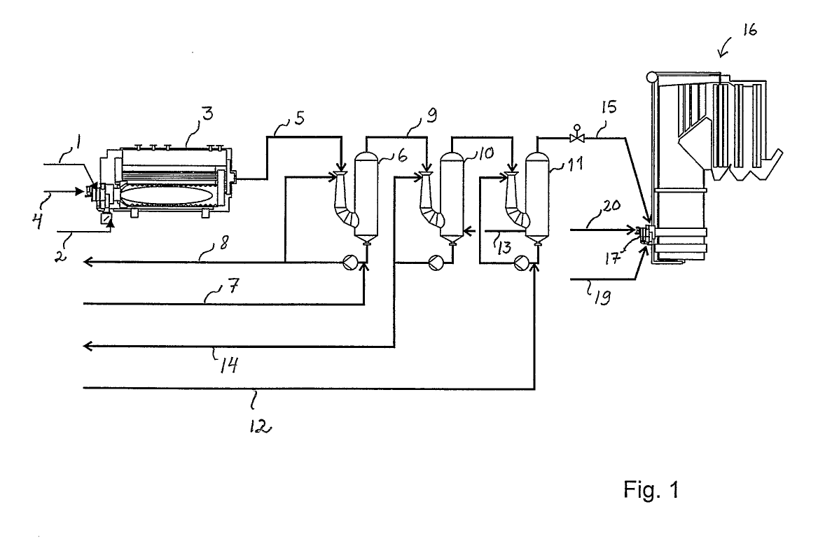

FIG. 1 illustrates the treatment of flue gas generated in the combustion of odorous gases. Strong odorous gases 1 are led into a separate combustion device 3, which typically is a fire tube boiler. Also air 2 and other required substances 4 are led into the combustion. The flue gas generated in the combustion is led via line 5 to scrubbing, where in the first stage the flue gas is scrubbed in a Venturi scrubber 6 with a sodium hydroxide containing solution 7. The sulfur dioxide contained in the flue gas reacts with sodium hydroxide, whereby sodiumbisulfite is formed and the solution 8 containing sodiumbisulfite can be used in the processes of the chemical pulp mill, for instance as anti-chlor in pulp bleaching. The bisulfite solution amount required at the mill can preferably be produced in the first flue gas scrubbing stage.

From the first scrubber 6 the flue gas is led via line 9 into two subsequent scrubbers 10 and 11 of the following scrubbing stage. A scrubbing solution 12, preferably oxidized white liquor, binding the sulfur compounds of the flue gas is led in the flue gas flow direction into the latter scrubber 11. From there the scrubbing solution is led via line 13 directly to the preceding scrubber 10, wherefrom the sodium sulfite containing scrubbing solution is led via line 14 e.g. into a white liquor tank (not shown).

The scrubbed flue gas is led in the flue gas flow direction from the last scrubber 11 via line 15 into the recovery boiler 16. In the embodiment according to the FIGURE a burner 17 has been installed in a wall of the recovery boiler, into which burner air via line and e.g. methanol via line 20 are led in addition to the scrubbed flue gas.

An advantage of the present invention is that the NOx in the flue gas of the separate combustion of odorous gases is not released into the surrounding atmosphere, but the scrubbed flue gas is led into the recovery boiler to be treated therein. The NOx-content of the recovery boiler does not increase at all, or at least does not substantially increase, although flue gases are fed into the boiler. Total emissions from a pulp mill in view of NOx can even be reduced compared to a situation, wherein a scrubbed flue gas of the separate combustion has been led to a chimney.

1. A method for treating odorous gases in a pulp mill comprising:
combusting the odorous gases in a separate combustion device, scrubbing the flue gas generated from the combustion of the odorous gas, and feeding the scrubbed flue gas to a recovery boiler.

2. The method of claim 1 wherein the scrubbed flue gas is fed to recovery boiler via a burner in a wall of the recovery boiler.

3. The method of claim 1 wherein the scrubbed flue gas is fed directly into the recovery boiler.

4. The method according to claim 4 wherein the flue gas is scrubbed in at least two stages.

5. The method of claim 4 wherein the flue gas is scrubbed in a first stage of the at least two stages with a sodium hydroxide-containing solution.

6. The method according to claim 1 wherein the flue gas is scrubbed in a second stage of the at least two stages wherein the second stage removes for removing sulfur compounds includes at least two scrubbers wherein scrubbed flue gas from a first scrubber in the second stage flows to a second scrubber in second stage, and the scrubbed flue gas from the second scrubber in the second stage is fed to the recovery boiler.

7. The method of claim 6 wherein the flue gas is scrubbed in the first scrubber of the second stage with oxidized white liquor.

8. The method of claim 5 wherein a sodiumbisulfite containing solution generated in the first stage is used at the chemical pulp mill.

9. The method of claim 7 wherein scrubbing solutions discharged from the second scrubber of the second stage is fed to the first scrubber of the second stage.

10. A method treating odorous gases in a pulp mill comprising:
feeding the odorous gas to a combustion device and combusting the odorous gas in the combustion device;
scrubbing in a first scrubbing stage flue gas generated in the combustion device from the combustion of the odorous gas and discharging scrubbed flue gas from the first scrubbing stage;
feeding the scrubbed flue gas from the first scrubbing stage to a second scrubbing stage;
scrubbing in the second scrubbing stage the scrubbed flue gas from the first scrubbing stage and discharging scrubbed flue gas from the second scrubbing stage, and
feeding the scrubbed flue gas discharged from the second scrubbing stage to a recovery boiler.

11. The method of claim 10 wherein the scrubbing in the first scrubbing stage includes scrubbing of the flue gas with a solution containing sodium hydroxide.

12. The method according to claim 10 wherein the second scrubbing stage includes a first scrubber and a second scrubber, and scrubbed flue gas from the first scrubbing stage is scrubbed in the first scrubber and scrubbed flue gas discharged from the first scrubber is fed to the second scrubber, and
scrubbing solution discharged from the second scrubber is fed to the first scrubber and used for scrubbing in the first scrubber.

13. The method of claim 12 wherein the flue gas is scrubbed in the first scrubber with oxidized white liquor.

14. A system to treat odorous gas in a pulp mill comprising:
a combustion device having an input receiving the odorous gas, in which the odorous gas is combusted, and having a discharge for flue gas generated by the combustion of the odorous gas;
a first stage of a scrubbing unit having a gas inlet receiving the flue gas from the combustion device and a gas outlet discharging scrubbed flue gas generated in the first stage, and
a second stage of a scrubbing unit having a gas inlet receiving the scrubbed flue gas from the first stage and a gas outlet for scrubbed flue gas from the second stage to be fed to a recovery boiler.

15. The system of claim 14 wherein the combustion device is a fire tube boiler.

16. The system of claim 14 wherein the second stage of the scrubbing unit includes a first scrubber and a second scrubber, wherein the first scrubber receives the scrubbed flue gas from the first stage and the second scrubber receives scrubbed flue gas from the first scrubber, and the scrubbed flue gas from the second scrubber is fed to the recovery boiler.

17. The system of claim 16 wherein the second scrubber includes a discharge for scrubbing solution which is coupled to an inlet for scrubbing solution to the first scrubber.

18. The system of claim 14 wherein a gas discharge outlet of the second stage is coupled to fed the scrubbed flue gas to a burner in a wall of the recovery boiler.

19. The system of claim 14 wherein the first stage has a solution inlet coupled to a solution source including sodium hydroxide.

20. The system of claim 14 wherein the second stage has a solution inlet coupled to a solution source including oxidized white liquor.
